(19) United States
(12) Plant Patent Application Publication
(10) Pub. No.: US 2017/0034974 P1
(43) Pub. Date: Feb. 2, 2017

(54) Apple tree named 'RKD'

(50) ;

(71) Applicant: Callahan Orchards LLC, Royal City, WA (US)

(72) Inventor: Richard Callahan, Royal City, WA (US)

(73) Assignee: Callahan Orchards LLC, Royal City, WA (US)

(21) Appl. No.: 14/756,078

(22) Filed: Jul. 28, 2015

(51) Int. Cl. A01H 5/00

(52) U.S. Cl. PLT/162

(57) ABSTRACT

‘RKD’ is a Gala-type apple tree distinguished by its early maturing and early full-coloring fruit.

### LATIN NAME

Malus domestica Borkh

### VARIETY DENOMINATION:

‘RKD’

### BACKGROUND OF THE VARIETY:

‘RKD’ is a new and distinct variety of apple tree Malus domestica Borkh that originated as a spontaneous two-limb mutation of ‘Tenroy’ gala (U.S. Plant Pat. No. 4,121). ‘RKD’ was discovered by the inventor in a commercial apple orchard in Royal City, Wash. in 2006, and was selected for further observation because of its distinctive early maturing and early full-coloring fruit. Buds were taken from the distinctive limbs in 2007 and budded onto rootstock for further observation and evaluation. The variety has since been reproduced by budding at Royal City, Wash., and has been observed to remain true to type through successive asexually propagated generations.

### BRIEF DESCRIPTION OF THE VARIETY

‘RKD’ is a Gala-type apple tree distinguished by its early maturing and early full-coloring fruit.

### BRIEF DESCRIPTION OF THE PHOTOGRAPHS

FIG. 1 shows a blossom, buds and young leaves on an ‘RKD’ apple tree;

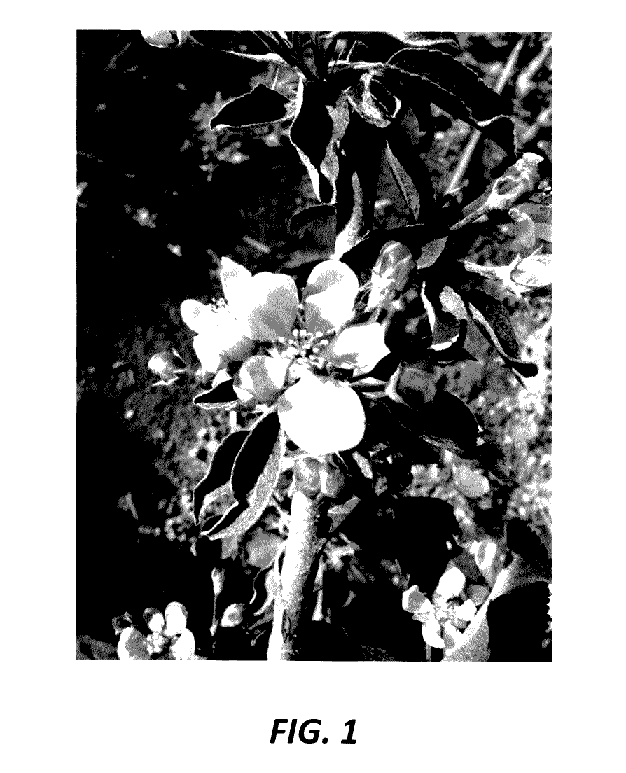

FIG. 2 shows the upper and lower surfaces of a leaf from an ‘RKD’ apple tree;

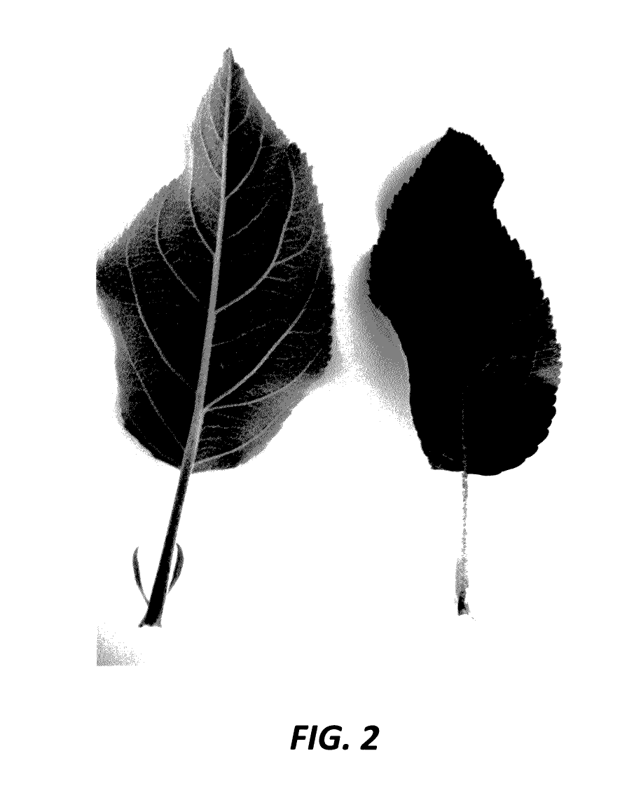

FIG. 3 shows fruit and leaves on an ‘RKD’ apple tree;

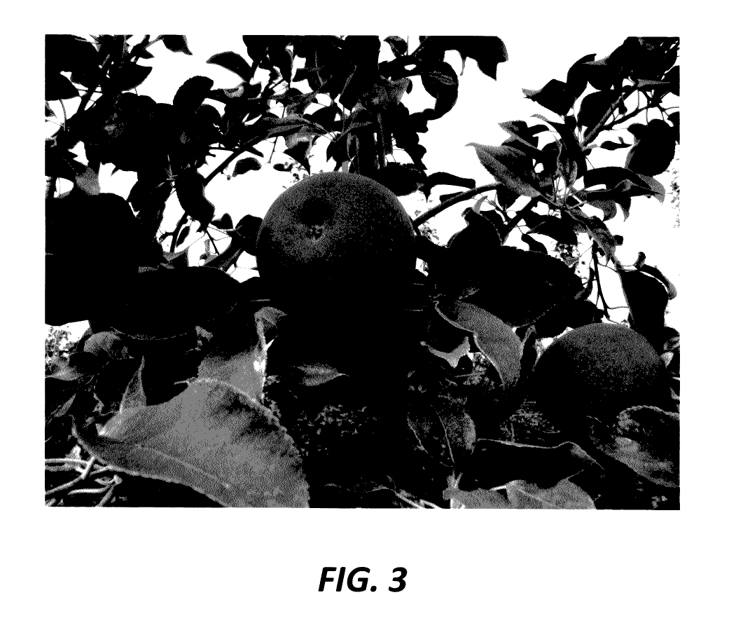

FIG. 4 shows a single ‘Simmons Gala’ apple next to ‘RKD’ apples on the tree; and

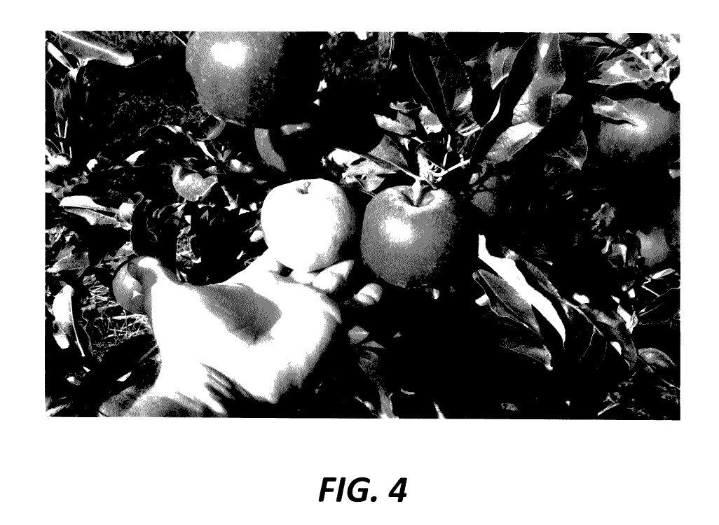

FIG. 5 shows a starch (maturity) test comparison of ‘RKD’ apples (left) and ‘Simmons Gala’ apples (right).

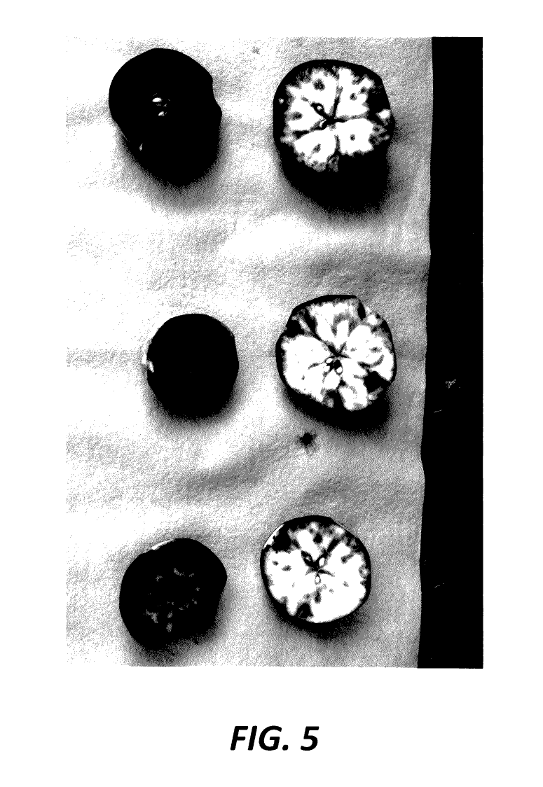

### DETAILED BOTANICAL DESCRIPTION OF THE VARIETY

The following-detailed botanical description is based on observations made during the 2013 and 2014 growing seasons at Royal City, Wash. of trees growing on ‘Cepiland’ rootstock (U.S. Plant Pat. No. 7,715), planted in 2011. All colors are described according to The Royal Horticultural Society Colour Chart. It should be understood that the characteristics described will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average.
Tree:
   Vigor.—Moderate.
   Type.—Non-spur.
   Habit.—Moderate spreading.
   Height.—2.4 m.
   Trunk diameter (at 30 cm above the graft).—3.3 cm.
   Bark texture.—Smooth.
   Bark color.—Brown 200C.
Branch (fruiting branches located at around 1 m above the graft union):
   Length.—73 cm.
   Diameter.—16 mm.
   Crotch angle.—60 degrees from vertical.
   Bark color.—Brown 200C.
   Lenticel length.—1 mm.
   Number of lenticels per cm².—5.
One year old shoot:
   Color.—Brown 200D.
   Pubescence.—Moderate.
   Thickness.—9 mm to 11 mm.
   Internode.—length 30 mm.
   Pubescence.—Slight.
   Number of lenticels per cm².—4.
Flower buds:
   Quantity per spur.—5.
   Shape.—Oblong, rounded.
   Length.—7 mm.
   Diameter.—5 mm.
   Color.—Red-purple N57B.
Flowers:
   Diameter of fully open flower.—Medium (king bloom).
   Relative position of petal margin.—Touching to overlapping.
   Number per cluster.—5 to 6.
   Date of first bloom.—Midseason, 3 days after ‘Simmons Gala’ (U.S. Plant Pat. No. 10,840).
   Date of full bloom.—4 days after ‘Simmons Gala’.
   Pollination requirement.—Pollinator required.
Petals:
   Number per flower.—5.
   Shape.—Orbicular.
   Length.—2.2 to 3.0 cm.
   Width.—1.7 cm.
   Apex.—Obtuse.
   Base.—Pointed.
   Margin.—Smooth.
   Coloration of upper surface.—White N155D.
   Coloration of lower surface.—White N155C.
Pistils:
   Size.—Medium.
   Color.—Yellowish green 145C.
Stigma:
   Size.—Small.
   Color.—Yellow-green 150B.
Style:
   Size.—Medium.
   Color.—Yellow-green 145C.
Ovary:
   Size.—Small, 0.4 mm.
   Color.—Yellow-green 145B.
Anthers:
   Quantity.—15 to 17.
   Size.—Medium, 2 mm.
   Presence of pollen.—Present.
   Color of pollen.—Yellow 3B.
Pedicel:
   Length.—27 mm.
   Diameter.—1.8 to 2.4 mm.
   Color.—Yellow-green 145B.
Sepals:
   Quantity.—5.
   Coloration.—Yellow-green 144C.
   Shape.—Pointed.
   Margin.—Smooth.
Leaves:
   Shape.—Ovate.
   Length.—101 mm.
   Width.—60 mm.
   Blade margin.—Serrate.
   Apex.—Acuminate.
   Base shape.—Oblique.
   Color of upper surface.—Green 143B.
   Color of lower surface.—Green 141A.
   Attitude in relation to shoot.—Outward to upward.
Petiole:
   Length.—3.1 cm.
   Diameter.—2 mm.
   Coloration.—Green 141C.
Fruit:
   Quantity per cluster.—1 to 2.
   Diameter.—75 mm.
   Weight.—203 g.
   Ratio of height to width.—Equal.
   General shape in profile.—Round to oblate.
   Position of maximum diameter.—Middle.
   Ribbing.—Slight.
   Crowning at calyx end.—Moderate.
   Size of eye.—1.1 cm.
   Aperture of eye.—Closed to partially open.
   Length of sepal.—5 mm.
   Bloom of skin.—Very slight.
   Greasiness of skin.—Very weak.
   Background color of skin.—Yellow 4B.
   Amount of over color.—90% or more.
   Over color of skin.—Red 46C.
   Intensity of over color.—Strong.
   Pattern of over color.—Blush over light striping.
   Amount of russet around stalk cavity.—Minimal.
   Amount of russet on cheeks.—None.
   Area of russet around eye basin.—None.
   Length of stalk.—1.8 mm to 3.0 cm.
   Thickness of stalk.—2.8 mm.
   Depth of stalk cavity.—17 mm.
   Width of stalk cavity.—Narrow, 10 mm.
   Depth of eye basin.—7.5 mm.
   Width of eye basin.—2.0 mm to 2.2 mm.
   Firmness of flesh.—Firm, 18+lbs. at harvest.
   Flesh texture.—Fine, crisp.
   Aroma.—Somewhat aromatic.
   Juiciness.—High.
   Flesh coloration.—Yellow 11D.
   Stem coloration.—Red 53C.
Seeds:
   Quantity per fruit.—1 to 2 per cell, 5 to 8 total.
   Shape.—Tear drop.
   Coloration.—Greyed-orange 164A.
Harvest:
   Time for harvest.—Aug. 3; 21+days before ‘Simmons Gala’.
   Number of picks.—1.
   Amount of fruit produced per tree per harvest.—36 lbs. per tree (1500 trees/acre).
   Market use.—Fresh market.

I claim:

1. A new and distinct apple tree substantially as described and illustrated herein.
